(19) United States
(12) Patent Application Publication
(10) Pub. No.: US 2026/0066096 A1
(43) Pub. Date: Mar. 5, 2026

(54) Displaying Healthcare Study Information

(71) Applicant: FUJIFILM Healthcare Americas Corporation, Lexington, MA (US)

(72) Inventors: Jasmine Mulligan, Seattle, WA (US); Prajeesh Prabhakaran, Morrisville, NC (US); Myles O’Keefe, Cary, NC (US)

(73) Assignee: FUJIFILM Healthcare Americas Corporation, Lexington, MA (US)

(21) Appl. No.: 18/820,423

(22) Filed: Aug. 30, 2024

(51) Int. Cl. G16H 30/40; G06T 7/00; G06T 11/60

(52) U.S. Cl. CPC G16H 30/40; G06T 7/0012; G06T 11/60; G06T 2200/24; G06T 2207/30068; G06T 2210/41

(57) ABSTRACT

Systems and methods for displaying healthcare study information are disclosed. In an implementation, a medical image management system receives a healthcare study from a first data source, generates study display layouts, and displays the layouts in a graphical user interface. The system determines whether a second data source includes automated image analysis findings for the healthcare study. In an instance where the second data source includes automated image analysis findings for the healthcare study, the system generates and displays automated image analysis display layouts.

### BACKGROUND

To diagnose patients, medical professionals (e.g., doctors) often order imaging studies (e.g., healthcare studies) that generate medical image data, which may be utilized to create an interpretation report. For certain types of screening studies (e.g., mammography, chest computed tomography (CT) scans), a double-reading protocol is utilized to generate the interpretation report. In some jurisdictions, the double-reading protocol involves the utilization of multiple readers (e.g., two clinicians) who independently review and interpret the healthcare study information. To avoid bias, the two readers are not allowed to see each other's findings before they generate their independent reports. In other jurisdictions, instead of utilizing a “two reader” double-reading protocol, the medical images are analyzed via computer-aided diagnosis (CAD) (e.g., computer-aided detection (CADe)) and CAD analysis results (e.g., annotated imaging data) are generated. The annotated imaging data is then considered by a single reader (e.g., clinician) in their review of the healthcare study information to generate the interpretation report. For a traditional medical image management system that implements computer-aided detection (CADe), the annotated imaging data is displayed (e.g., as an overlay) for review by the clinician during each review step. However, displaying annotated imaging data to the clinician before the clinician makes their initial diagnosis may bias the clinician's diagnosis.

### SUMMARY

Systems and methods for displaying healthcare study information are disclosed. In aspects, the techniques disclosed herein may implement a computer-aided diagnosis integrated (CAD-integrated) reading protocol to present study images and other relevant medical imaging data (e.g., legacy imaging data, annotated imaging data) in a graphical user interface (GUI) of a display device (e.g., a display monitor). The CAD-integrated reading protocol may define a first set of review steps (e.g., study review steps) where a first series of medical images (e.g., study images) are displayed for review by a clinician (e.g., radiologist). During the first set of review steps, annotated imaging data associated with the study images is not displayed to the clinician.

Upon completion of a last review step of the first set of review steps, if annotated imaging data exists for the healthcare study, a second set of one or more (CAD) review steps (e.g., CAD review steps) is automatically advanced to (e.g., without the clinician providing separate user input to show CAD images). In the second set of review steps, one or more of the study images are displayed in the GUI with the annotated imaging data (overlay) turned ON. After advancing through the last review step of the second set of review steps, the CAD-integrated reading protocol ends. Upon completion of a last review step of the first set of review steps, if there is no annotated imaging data for the case (e.g., no automated image analysis findings, was not analyzed by computer-aided diagnosis), then the second set of review steps may be omitted and the CAD-integrated reading protocol ends. In this way, the problem of biasing a clinician's independent judgment by prematurely displaying annotated imaging data is solved by an improved reading protocol where the review steps that include the annotated imaging data are displayed after the clinician has completed their initial review of the study images. Further, the improved reading protocol improves the clinician's user experience, enabling the clinician to work more efficiently, which improves the timeliness of patient care.

In some aspects, the techniques described herein relate to a method including receiving a healthcare study from a first data source, the healthcare study having a first image series that includes more than one image; generating, from the first image series, study display layouts for display in a graphical user interface of a display device, the study display layouts including a first display layout and a last display layout; displaying the first display layout in the graphical user interface; receiving a user input to advance to a next display layout; displaying the last display layout in the graphical user interface; receiving a further user input to advance to a next display layout; and determining whether a second data source includes automated image analysis findings for the healthcare study. In an instance where the second data source includes automated image analysis findings for the healthcare study, the method further including generating an automated image analysis display layout for display in the graphical user interface from the automated image analysis findings; and displaying the automated image analysis display layout in the graphical user interface on the display device after the displaying of the last display layout of the first image series of the healthcare study. In an instance where the second data source does not include automated image analysis findings for the healthcare study, the method further including displaying a notification to a user in the graphical user interface that indicates that there are no automated image analysis findings for the healthcare study.

### BRIEF DESCRIPTION OF THE DRAWINGS

The appended drawings illustrate examples of systems and methods for displaying healthcare study information and are, therefore, not considered to be limiting in scope.

FIG. 1 illustrates an example environment for a medical image management system, with which aspects of the disclosed systems and methods for displaying healthcare study information may be implemented.

FIG. 2 is a block diagram showing an example of a computing system architecture for displaying healthcare study information.

FIG. 3 illustrates an example process for displaying healthcare study information.

FIG. 4 illustrates an example process for displaying healthcare study information.

FIGS. 5A and 5B depict a method for displaying healthcare study information, in accordance with one or more implementations.

FIG. 6 illustrates an example aspect of a logical representation of a medical image management system that displays healthcare study information.

### DETAILED DESCRIPTION

Disclosed are systems and methods for displaying healthcare study information. A medical image management system may generate (e.g., by a healthcare modality of the medical image management system) healthcare study information. The healthcare study information includes medical imaging data (e.g., study images) for a current healthcare study and may also include one or more of legacy medical imaging data (e.g., study images from a previous healthcare study) or annotated imaging data.

The medical image management system may utilize computer-aided diagnosis (CAD) (e.g., computer-aided detection (CADe)) to perceive and mark latent features (e.g., potential abnormalities) within the medical imaging data for review by a clinician. Examples of latent features include microcalcifications, masses, and physical characteristics (e.g., sphericity). Through utilization of computer-aided diagnosis, locations of latent features in the medical imaging data may be determined to generate annotated imaging data. In one example of annotated imaging data, a study image is annotated with indicia (e.g., one or more tags, icons, arrows, pointers) that mark the location(s) of a latent feature to generate an annotated image. In another example of annotated imaging data, an overlay that includes indicia is generated for display on top of a study image.

The healthcare study information may be displayed by the medical image management system on a display device (e.g., in a graphical user interface (GUI)) of a display component for review by the clinician. For example, one or more study images may be displayed on the display device alongside annotated imaging data, one or more study images overlaid with annotated imaging data may be displayed on the display device, annotated images may be displayed on the display device, and the like.

The clinician may utilize the display component to analyze the medical image data. The display component renders the images in display layouts (layouts) to implement a CAD-integrated reading protocol. A display layout may include different combinations of the medical image data (e.g., a split screen of an old healthcare study and the current healthcare study, different sides of a breast). For example, a first layout may include current and legacy mediolateral oblique (MLO) projections, a second layout may include current and legacy cranialcaudal (CC) projections, a third layout may include both current projections (e.g., the MLO and the CC), and the like. Display layouts may be displayed on the same display component and/or elements of a layout may be displayed (e.g., side-by-side) on multiple display components.

The clinician may provide a user input to advance through the review steps (e.g., to advance to a next display layout). The medical image management system is configured to receive user input from the clinician through an input device (e.g., a keyboard, a keypad, a pointing device (commonly referred to as a mouse), a trackball, a touch pad, a microphone, a scanner, a motion sensor, and the like). The user input may include a “command” invoked by the clinician to advance to a next review step (e.g., display layout, image) in the CAD-integrated reading protocol (e.g., a button press, a keyboard press, a mouse click, a voice input, and the like).

In view of the study images, the clinician reviews and interprets the annotated imaging data to determine if they include information that is relevant to the clinician's diagnosis, which thereby enables the clinician to consider their diagnosis in view of the annotated imaging data. In this way, computer-aided diagnosis may be utilized to replace and/or supplement the traditional double-reading protocol review by two individual clinicians in a screening room.

In the following description, details are set forth to provide a more thorough explanation of the disclosed systems and methods for displaying healthcare study information, which may be implemented by a medical image management system. It will be apparent, however, to one skilled in the art, that the disclosed systems and methods may be practiced without these specific details. In other instances, well-known structures and devices are shown in block diagram form, rather than in detail, in order to avoid obscuring the disclosed systems and methods. The subject matter of aspects of the disclosed systems and methods for displaying healthcare study information is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies.

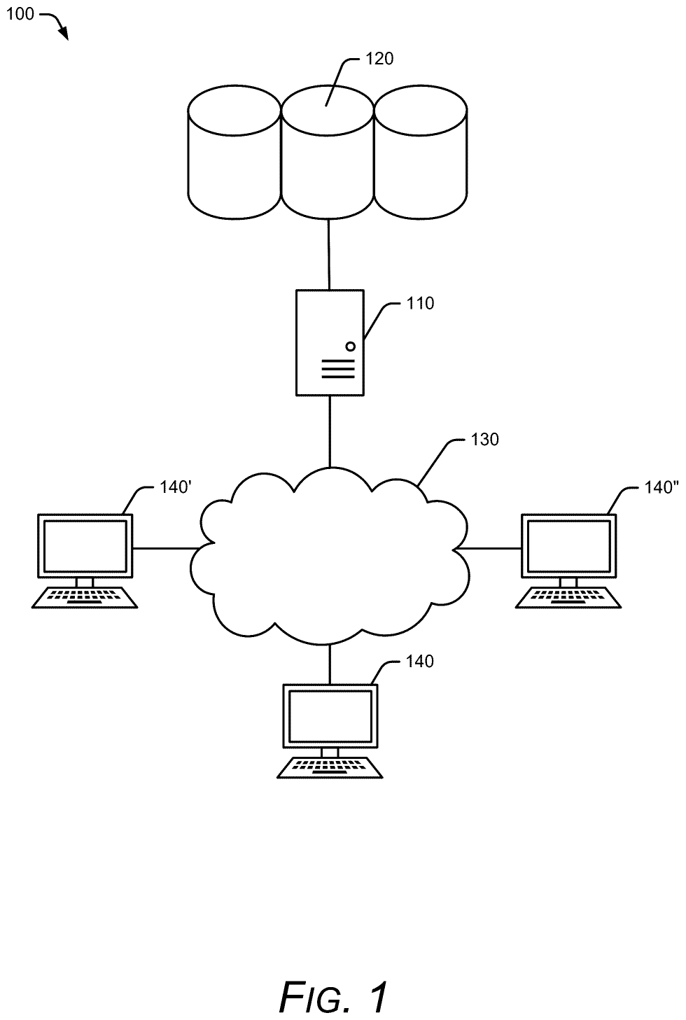

Having briefly described an overview of the disclosed systems and methods for displaying healthcare study information, aspects will be discussed with reference to FIGS. 1-6.

### Environment

Referring to the drawings in general, and initially to FIG. 1 in particular, a medical image management system environment 100, with which aspects of the disclosed systems and methods may be implemented, is illustrated. It will be understood and appreciated by those of ordinary skill in the art that the illustrated environment 100 is merely an example of one suitable computing environment and is not intended to suggest any limitation as to the scope of use or functionality of the disclosed systems and methods. Neither should the environment 100 be interpreted as having any dependency or requirement relating to any single component or combination of components illustrated therein. The disclosed systems and methods may be operational with numerous general-purpose or special-purpose computing system environments or configurations. Examples of well-known computing systems, environments, and/or configurations that may be suitable for use with the disclosed systems and methods include, by way of example only, personal computers, server computers, hand-held devices, laptop devices, multiprocessor systems, microprocessor-based systems, programmable consumer electronics, network personal computers, minicomputers, mainframe computers, distributed computing environments that include any of the above-mentioned systems or devices, and the like.

Aspects of the disclosed systems and methods may be described in the general context of computer-executable instructions (e.g., program modules) configured for execution by a computer. Generally, program modules include, but are not limited to, routines, programs, objects, components, and data structures that perform particular tasks or implement particular abstract data types. The disclosed systems and methods may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the example medical image management system environment 100 includes a general-purpose computing device in the form of a control server 110. Components of the control server 110 may include, without limitation, a processing unit, internal system memory, and a suitable system bus for coupling various system components, including a database cluster 120, with the control server 110. The system bus may be any of several types of bus structures, including a memory bus or memory controller, a peripheral bus, and a local bus, using any of a variety of bus architectures. By way of example, and not limitation, such architectures include the Industry Standard Architecture (ISA) bus, the Micro Channel Architecture (MCA) bus, the Enhanced ISA (EISA) bus, the Video Electronic Standards Association (VESA) local bus, and the Peripheral Component Interconnect (PCI) bus, also known as a Mezzanine bus.

The control server 110 may include therein, or have access to, a variety of computer-readable media (e.g., the database cluster 120). Computer-readable media can be any available media that may be accessed by the control server 110 and include volatile and non-volatile media, as well as removable and non-removable media. By way of example, and not limitation, computer-readable media may include computer storage media. Computer storage media may include, without limitation, volatile and non-volatile media, as well as removable and non-removable media implemented in any method or technology for storage of information, such as computer-readable instructions, data structures, program modules, or other data. In this regard, computer storage media may include, but are not limited to, random-access memory (RAM), read-only memory (ROM), electrically erasable programmable read-only memory (EEPROM), flash memory or other memory technology, compact disc read-only memory (CD-ROM), digital versatile disks (DVDs) or other optical disk storage, magnetic cassettes, magnetic tape, magnetic disk storage, or another magnetic storage device, or any other medium that can be used to store the desired information and may be accessed by the control server 110. By way of example, and not limitation, communication media include wired media such as a wired network or direct-wired connection and wireless media such as acoustic, radio frequency (RF), infrared, and other wireless media. Combinations of any of the above also may be included within the scope of computer-readable media.

The computer storage media discussed above and illustrated in FIG. 1, including the database cluster 120, provides storage of computer-readable instructions, data structures, program modules, and other data for the control server 110. The control server 110 may operate on a computer network 130 using logical connections to one or more remote computers (e.g., remote computer 140, remote computer 140′, remote computer 140″). A remote computer may be located at a variety of locations in a medical or research environment, including, but not limited to, clinical laboratories (e.g., molecular diagnostic laboratories), hospitals and other inpatient settings, veterinary environments, ambulatory settings, medical billing and financial offices, hospital administration settings, home health care environments, and clinicians' offices. A remote computer may also be physically located in non-traditional medical care environments so that the entire healthcare community may be capable of integration into the network. A remote computer may be personal computers, servers, routers, network personal computers, peer devices, other common network nodes, or the like, and may include some or all of the elements described above in relation to the control server 110. The devices can be personal digital assistants or other like devices.

An example computer network 130 may include, without limitation, local area networks (LANs) and/or wide area networks (WANs). Such networking environments are commonplace in offices, enterprise-wide computer networks, intranets, and the Internet. When utilized in a WAN networking environment, the control server 110 may include a modem or other means for establishing communications over the WAN, such as the Internet. In a networked environment, program modules or portions thereof may be stored in association with the control server 110, in association with the database cluster 120, or in association with one or more of the remote computers (e.g., remote computer 140). For example, and not by way of limitation, various application programs may reside on a memory associated with any one or more of the remote computers. It will be appreciated by those of ordinary skill in the art that the network connections shown are examples and other means of establishing a communications link between the computers (e.g., control server 110 and remote computer 140) may be utilized.

Clinicians may include, but are not limited to, a treating physician or physicians, specialists such as intensivists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, radiologic technologists, researchers, veterinarians, students, and the like. In operation, a clinician may enter commands and information into the control server 110 or convey the commands and information to the control server 110 via one or more remote computers (e.g., remote computer 140, remote computer 140′, remote computer 140″) through input devices, such as a keyboard, a pointing device (commonly referred to as a mouse), a trackball, or a touch pad. Other input devices may include, without limitation, microphones, scanners, or the like. Commands and information may also be sent directly from a remote healthcare device to the control server 110. A control server 110 and/or a remote computer may include other peripheral output devices, such as speakers and a printer.

Although many other internal components of the control server 110 and remote computers (e.g., remote computer 140, remote computer 140′, remote computer 140″) are not shown, those of ordinary skill in the art will appreciate that such components and their interconnection are well known. Accordingly, additional details concerning the internal construction of the control server 110 and remote computers (e.g., remote computer 140) are not further disclosed herein.

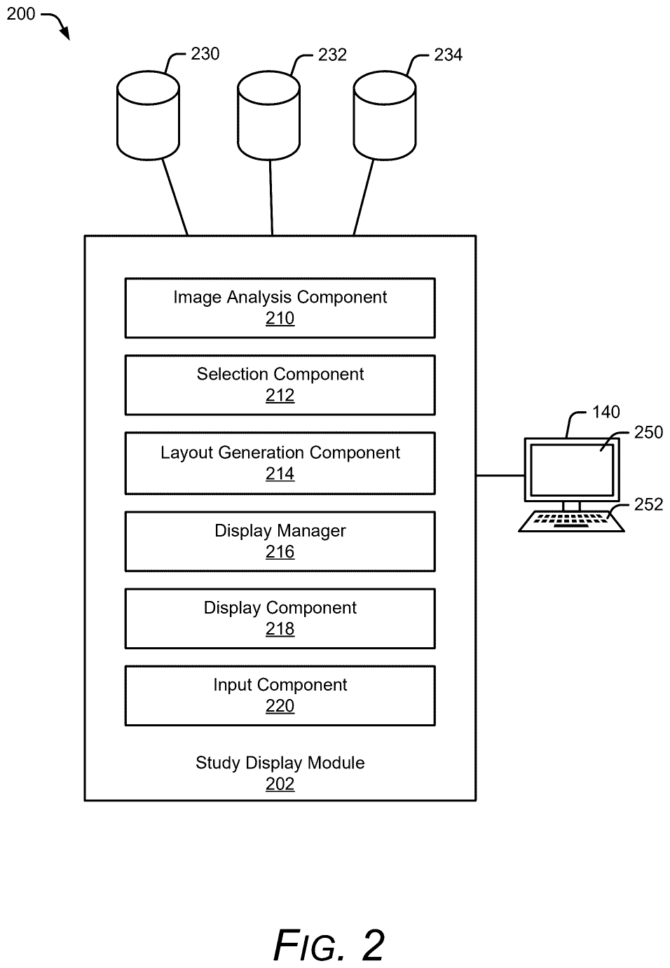

With reference to FIG. 2, a block diagram is illustrated that shows an example of a computing system architecture for displaying healthcare study information (e.g., medical images), for example, on a display device of a display component. It will be appreciated that the computing system architecture for a computing system 200 illustrated in FIG. 2 is merely an example of one suitable computing system and is not intended as having any dependency or requirement related to any single module/component or combination of modules/components.

In one aspect, the computing system 200 includes a study display module 202 and one or more data sources (e.g., database cluster 120), which include at least one image data set of medical images for a healthcare study (e.g., projection X-ray images, synthetic images, non-synthetic images, tomosynthesis projection images related to the projection X-ray images, thumbnails, and the like). In an example, the data sources may include a database 230 that stores and maintains current healthcare studies that contain current medical images, a database 232 that stores and maintains existing (previous) healthcare studies, and/or a database 234 that stores and maintains findings (“automated image analysis findings”) that result from application of one or more automated image analysis algorithms (e.g., artificial intelligence (AI) analysis algorithms) to images of the current healthcare studies (e.g., those stored in database 230). A data source may contain images or other study data (e.g., medical parameter values) that are linked to a patient's electronic medical record (EMR). As utilized herein, the acronym “EMR” is not meant to be limiting and may broadly refer to any or all aspects of the patient's medical record rendered in a digital format. Generally, the EMR is supported by systems configured to coordinate the storage and retrieval of individual records with the aid of computing devices. As such, a variety of types of healthcare-related information may be stored and accessed in this way. One or more of the data sources may be maintained separately. Two or more of the data sources may be integrated. In aspects, the data sources (e.g., database 230, database 232, database 234) may be a picture archiving and communication system (PACS), a vendor neutral archive (VNA), other repository systems, or other database systems. The data sources may be spread across multiple facilities and/or multiple locations. A database may include one or more databases.

In one aspect, the healthcare studies (e.g., current healthcare studies, existing healthcare studies) include medical images and study data (e.g., healthcare study information). The healthcare study information may include values of one or more medical parameters (e.g., parameter values, measurements, findings, impressions, patient demographics and history/risk factors) related to the healthcare study. Examples of medical images include radiology images (e.g., mammography images), laboratory images, pictures, cardiology images (e.g., echocardiography images), and other healthcare images (e.g., medical image data). A healthcare study may include one or more series of one or more medical images. For example, a healthcare study may have a first image series that includes more than one image.

The study display module 202 may include one or more of an image analysis component 210, a selection component 212, a layout generation component 214, a display manager 216, a display component 218, and an input component 220. The study display module 202 may reside on one or more computing devices (e.g., the control server 110 described above with reference to FIG. 1). By way of example, in one aspect, the control server 110 includes one or more computer processors and may be a server, personal computer, desktop computer, laptop computer, handheld device, mobile device, consumer electronic device, or the like.

The image analysis component 210 (e.g., an AI analysis component) may perform image analysis on healthcare studies (e.g., medical image data stored in database 230) to produce automated image analysis findings (e.g., results, outputs) that are sent for clinician review and/or are stored in a data source (e.g., database 234) for subsequent access. The image analysis may include activation of one or more automated image analysis algorithms that execute on one or more artificial intelligence (AI) engines and/or servers (e.g., control server 110 described above with reference to FIG. 1) to produce results that are indicative of the findings of the algorithm. The findings may include images, or portions thereof, from the analyzed healthcare study that are relevant to the diagnosis or condition of the patient.

The image analysis component 210 may analyze medical imaging data and mark perceived latent features in the medical imaging data to generate annotated imaging data for review by the clinician. In this way, the image analysis component 210 may generate a second image series of one or more annotated images from the automated image analysis findings. The second image series may include annotated imaging data associated with one or more images of the healthcare study (e.g., images of the first image series (e.g., mammography images)).

The image analysis by the image analysis component 210 may include a determination of the existence of automated image analysis findings for one or more images of the first image series. The image analysis component 210 may generate at least one automated image analysis finding indicator from the automated image analysis findings for images of the first image series that have automated image analysis findings. The image analysis component 210 may combine the generated automated image analysis finding indicator(s) with the respective images of the first image series to generate images for the second image series (e.g., generate CAD-generated images). The generation of the second image series from the automated image analysis findings may include the inclusion of copies of images of the first image series that do not have automated image analysis findings in the second image series.

The image analysis component 210 may mark perceived latent features in the medical imaging data through use of one or more indicia (e.g., tags, icons, arrows, pointers). The annotated imaging data may include a study image that is annotated with indicia to generate an annotated image, an overlay that includes indicia that is displayed on top of a study image, and the like. The annotated imaging data may include one or more study images with automated image analysis finding indicators (e.g., annotations) that mark one or more perceived latent features in the study images for review by a clinician. The annotated imaging data may include one or more image overlays based on one or more images of the first image series, and the image overlays may include automated image analysis finding indicators that are presented as an image overlay on the images of the first image series. In this way, the second image series may include at least one of first image views of one or more of the images in the first image series overlaid with the at least one automated image analysis finding indicator. The image analysis component 210 may generate a compiled image that overlays the automated image analysis finding indicators onto an analyzed image of the first image series.

The study display module 202 may receive healthcare study information (e.g., medical images) from a data source (e.g., a database, via a link within a patient's EMR). For example, the selection component 212 may select healthcare study information (e.g., a healthcare study) from a first data source and the study display module 202 may receive it. The healthcare study may include at least one of a current healthcare study or one or more previous healthcare studies. The healthcare study information may include an image data set of medical images for a healthcare study, which may include one or more series of one or more medical images. For example, the healthcare study information for a healthcare study may include a first image series that includes more than one image. The study display module 202 may also receive automated image analysis findings (e.g., from database 234) that result from application of one or more automated image analysis algorithms (e.g., automated image analysis algorithms) to images of the healthcare studies from a data source. The automated image analysis findings may include a second image series that includes more than one image. The selection component 212 selects healthcare studies and may access data sources (e.g., databases) to obtain the selected healthcare studies. In this way, the study display module 202 may receive the healthcare studies.

A selected healthcare study may include one or more pieces of information related to the healthcare study. The pieces of information may include, but are not limited or restricted to, (i) medical images (e.g., x-rays, mammograms, computerized tomography (CT) scans, magnetic resonance imaging (MRI), positron emission tomography (PET) scan, ultrasound imaging), (ii) clinician notes regarding one or more of the medical images, and/or (iii) medical records corresponding to one or more of subjects of the one or more medical images. The medical records may include other study data, for example medical parameter values (e.g., measurements, findings, impressions, patient demographics and history/risk factors) related to the healthcare study. A series of images may include one or more images that depict a subject of the image from various angles.

The selection component 212 may determine whether a data source (e.g., database 234) includes automated image analysis findings (e.g., annotated imaging data) for a healthcare study. The selection component 212 may select the automated image analysis findings (e.g., from database 234) that result from the application of the one or more automated image analysis algorithms to the images of the healthcare study. In this way, the study display module 202 may receive the automated image analysis findings.

Utilizing the received healthcare study information and the automated image analysis findings (e.g., CAD-generated images), if present (e.g., stored in a database), selected by the selection component 212, the layout generation component 214 may generate a display layout (e.g., layout) for display in a graphical user interface (GUI) on a display device of the display component 218. The generation of the display layout may include generation of study display layouts (e.g., a sequence of first image views of one or more of the images in the first image series) for display in the GUI. The study display layouts may include a first display layout and a last display layout.

A display layout may further include a sequence of second image views of the images in the second image series (e.g., an automated image analysis display layout generated from the automated image analysis findings). The layout generation component 214 may receive the automated image analysis findings for the healthcare study from a second data source. The first data source and the second data source may be the same data source. Displaying the automated image analysis findings for the healthcare study in the GUI after displaying of a last view of the first image series of the healthcare study may include displaying, by the display component 218, the second image views of the images in the second image series.

If the data source (e.g., database 234) does not include automated image analysis findings for the healthcare study, the display component 218 may display a notification to the user in the GUI that indicates that there are no automated image analysis findings for the healthcare study. The notification may be displayed after the display of the last view and/or with the display of the last view.

A study display layout may further include a display of one or more thumbnail images for the study to enable the radiologist to navigate the results of the study. The mammographic images for a study may be grouped together into series, with each series represented by a thumbnail image. The series grouped mammographic images may include images that have same position (e.g., CC, LMO) and side (e.g., left, right). When displayed in the GUI (e.g., within a user interface element), a thumbnail image may include a numeric indictor that represents the number of mammographic images that the thumbnail represents. The thumbnail image may be received from a data source (e.g., database cluster 120). The thumbnail may be generated by the layout generation component 214 from medical image data stored in a database (e.g., database 230).

The study display layout may include a user interface element (e.g., an image slider element) configured for display of the thumbnail images. In aspects, the image slider element may be one or more of an image slider, an image carousel, a carousel slider, an image slider, a slider bar, a slide panel, a gallery, a slideshow, and the like. The image slider element may be configured to move (e.g., slide, rotate) automatically for navigation, may be configured for manual navigation (e.g., move from left to right) by a user without navigating away from the view of study on the GUI, etc. A user may interact with the image slider element to navigate through (e.g., scroll through) the thumbnail images (e.g., by swiping on a touchscreen display, pushing arrow keys on a keyboard, using a mouse).

The display layout may allow one or more images of the current study to be displayed for review by the clinician. The display layout may allow one or more images of the current study to be compared to one or more images of at least one previous study. The display layout may allow one or more images of the current study to be compared to one or more images (e.g., annotated imaging data) generated by the layout generation component 214 that include automated image analysis findings for the current study. In one aspect, the layout generation component 214 creates a side-by-side layout with series of images of the current and previously created studies rendered next to each other within the display component. The display layouts may be ordered to present the first image series in a first set of review steps and present the second image series in a second set of review steps, with the first set of review steps presented before the second set of review steps. A display layout may define configurations including parts of the screen layout, display contexts (e.g., image data, layouts, arrangement of images, size of images), and the like to facilitate an ordered review process.

The display manager 216 (e.g., rendering component) receives the display layout and generates the GUI on the display device of the display component 218. The GUI may be implemented within an image viewer or an image viewer application of the display device. The GUI is configured to display the contents of the study, including any images and findings contained therein. The display layout may define a CAD-integrated reading protocol that includes multiple review steps that may include one or more images.

The display component 218 includes a display device (e.g., a monitor, a computer screen, a project device, other hardware device) for displaying in a GUI display layouts containing images and other data from healthcare studies and/or automated image analysis findings to images in the healthcare studies. For example, the remote computer 140 described above with reference to FIG. 1 may include a display component with a display device. The display component 218 displays the display layouts generated by the display manager 216 in the GUI of the display device (e.g., in a GUI 250 of the display device of the remote computer 140 illustrated in FIG. 2). The display layouts may contain images and other data from healthcare studies, as well as automated image analysis findings to images in the healthcare studies.

The display component 218 may also display the images of the second image series in the GUI. The second image series may include annotated imaging data, (e.g., a study image that is annotated with indicia to generate an annotated image, an overlay including indicia that is displayed on top of a study image, and the like). The display component 218 may display the second image series of the healthcare study in the GUI after the display of the last view of the first image series in the GUI.

The input component 220 receives a user input(s) from a user (e.g., a clinician), for example, a user input to advance to a next display layout displayed in the GUI. The user input may be received from an input device (e.g., a keyboard, a keypad, a mouse, and the like). In one example, the input device is a keyboard 252 of the remote computer 140. The user input received by the input component 220 may include a command invoked by the clinician to advance to a next review step (e.g., display layout) in the CAD-integrated reading protocol. Examples of a command invoked by a clinician include a button press, a keyboard press, a mouse click, a voice input, and the like.

Responsive to receiving a user input to advance to the next review step in the CAD-integrated reading protocol, the display manager 216 may change the GUI to display a next display layout (e.g., image, finding) of the healthcare study. Upon receiving a user input to advance to a next display layout after the last view of the first image series of the healthcare study has been displayed, the display manager 216 may change the GUI on the display device of the display component 218 to display a first display layout of the second series.

It is desirable to implement a CAD-integrated reading protocol that defines a first set of review steps configured to permit the clinician to review a series of medical images with an annotated imaging data overlay turned OFF and receive user input from the clinician to advance to the next review step (e.g., by invoking a command). Upon completion of a last review step of the first set of review steps, if annotated imaging data exists, a second set of one or more review steps may be automatically advanced to (e.g., without the clinician providing user input to show the annotated imaging data). In the second set of review steps, one or more of the study images are displayed in the GUI with the annotated imaging data (overlay) turned ON. After advancing past the last step of the second set of review steps, the CAD-integrated reading protocol ends. In implementations, the last step of the second set of review steps can be a repetition of the review steps of the first set of review steps with the annotated imaging data overlay turned ON. If there is no annotated imaging data for the case (e.g., no automated image analysis findings, was not analyzed by computer-aided diagnosis), then the last review step of the second set of review steps is omitted and the CAD-integrated reading protocol ends. Upon completion of the last step of the first set of review steps, if annotated imaging data does not exist, then the CAD-integrated reading protocol ends.

In this way, the problem of biasing a clinician's independent judgment by prematurely displaying annotated imaging data is solved by an improved CAD-integrated reading protocol where review steps that include the annotated imaging data are displayed after the clinician has completed their initial review of the study images. Further, the improved CAD-integrated reading protocol improves the clinician's user experience, enabling the clinician to work more efficiently, which improves the timeliness of patient care.

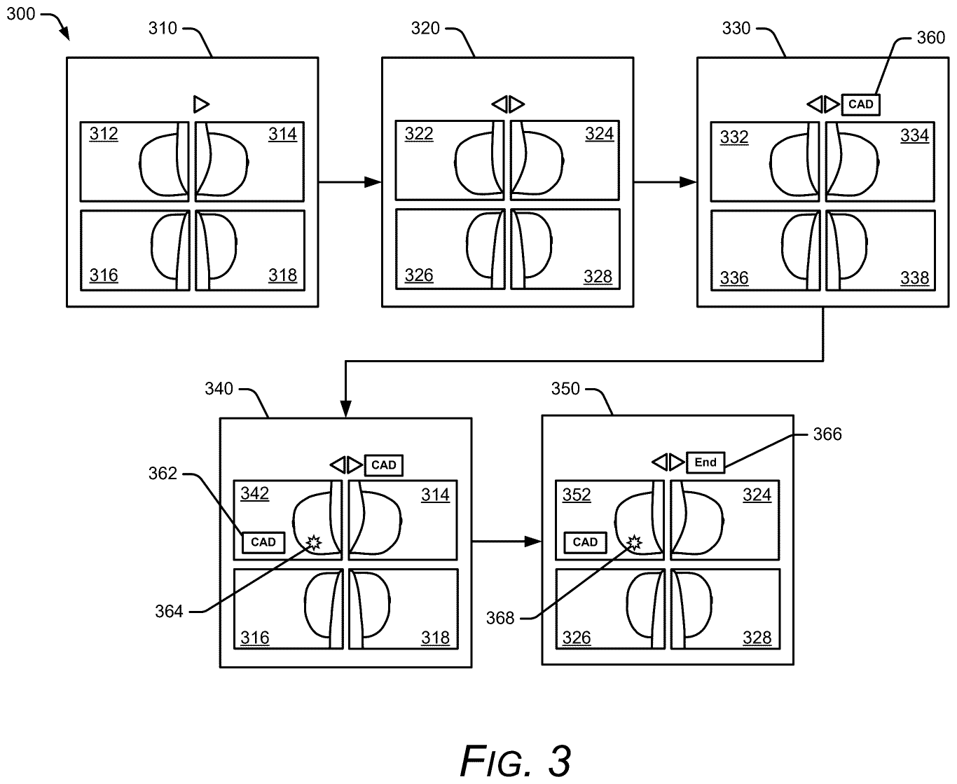

FIG. 3 illustrates an example process 300 for displaying healthcare study information, which may include medical imaging data from a current healthcare study and may also include one or more of legacy medical imaging data (e.g., study images from a previous healthcare study) or annotated imaging data. The process 300 is illustrated as a series of blocks that specify operations performed. The operations are not necessarily limited to the order or combinations shown for performing the operations by the respective blocks. Further, any of one or more of the operations can be repeated, combined, reorganized, or linked to provide a wide array of additional and/or alternate processes. In portions of the following discussion, reference can be made to the example system environment 100 of FIG. 1 and/or to entities or processes as detailed in FIGS. 1 and 2, reference to which is made for example only. The techniques are not limited to performance by one entity or multiple entities operating on one device. The operations illustrated and described herein are examples.

As discussed with respect to the computing system 200 of FIG. 2, a study display module (e.g., study display module 202) receives selected healthcare study information for a current healthcare study from a data source (e.g., first database 230). The healthcare study information is selected by a selection component (e.g., selection component 212) and has a first image series that includes more than one medical image. Utilizing the selected healthcare study information, a layout generation component (e.g., layout generation component 214) generates display layouts for displaying a sequence of first image views (e.g., a first image series) of one or more of the medical images (e.g., mammography images) of the current healthcare study from the first image series. For example, the study display layouts from the first image series may include a first display layout 310, a second (intermediate) display layout 320, and a last (third) display layout 330. While FIG. 3 only includes a single intermediate display layout 320 in the first image series, in implementations, additional intermediate image views may be represented in additional display layouts. Further, an intermediate view of the first image series may not be present.

The display layouts are for display in a GUI (e.g., GUI 250) of a display device of a display component (e.g., display component 218). The display layouts may include one or more current projections, legacy projections (e.g., legacy images from previous healthcare study for the patient), or annotated projections including annotated imaging data. A display layout may include more than one type of projection to provide a side-by-side comparison of projections.

In the example of FIG. 3, the first display layout 310 includes first current mediolateral oblique (MLO) projections and first current cranialcaudal (CC) projections (e.g., right cranialcaudal (RCC) projection 312, left cranialcaudal (LCC) projection 314, right mediolateral oblique (RMLO) projection 316, left mediolateral oblique (LMLO) projection 318). The second (intermediate) display layout 320 includes second current MLO projections and second current CC projections (e.g., RCC projection 322, LCC projection 324, RMLO projection 326, LMLO projection 328). The last display layout 330 includes third current MLO projections and third current CC projections (e.g., RCC projection 332, LCC projection 334, RMLO projection 336, LMLO projection 338). Again, these are merely examples of projections that may be present in a display layout for the study and, in aspects, more or fewer display layouts may be present for the first image views of the one or more of the medical images of the current healthcare study from the first image series.

A display manager (e.g., display manager 216) receives the display layout(s) and generates a graphical user interface (GUI) on a display device of a display component (e.g., display component 218), which displays the display layout on the display device. In this way, the first display layout 310 is displayed to the clinician for review. After reviewing the contents of the first display layout 310, the clinician provides (e.g., via input component 220) user input to advance the display to a next display layout. The display manager (e.g., display manager 216) then changes the GUI to display the next display layout of the healthcare study (e.g., the second (intermediate) display layout 320). The process of displaying a display layout on the GUI and receiving user input from the clinician to advance the display to a next display layout may iteratively proceed until the clinician has viewed all display layouts of the first image series of the healthcare study.

After reviewing the contents of the last display layout 330 for the first image series, the clinician may provide (e.g., via input component 220) further user input to advance the display to a next display layout. Upon receiving a user input to advance to a next display layout after the last display layout 330 of the first image series of the healthcare study has been displayed, the selection component (e.g., selection component 212) may determine whether a second data source includes automated image analysis findings for the healthcare study.

In an instance where the second data source includes automated image analysis findings for the healthcare study, the layout generation component (e.g., layout generation component 214) may generate one or more automated image analysis display layouts for display in the GUI that includes automated image analysis findings (e.g., a sequence of second image views of images in a second image series). For example, in the process 300, the example sequence of display layouts further includes automated image analysis display layouts (e.g., fourth display layout 340, fifth display layout 350) for the healthcare study.

The automated image analysis display layout may include annotated imaging data (e.g., medical imaging data in which findings (e.g., latent features) are marked). The annotated imaging data may include a study image that is annotated with indicia to generate an annotated image, an overlay including indicia that is displayed on top of a study image, and the like. For example, a fourth display layout 340 may include projections from another operation (e.g., the projections of display layout 310) overlaid with first findings (e.g., annotated imaging data), and a fifth display layout 350 may include projections of the display layout 320 overlaid with second findings (e.g., annotated imaging data). While FIG. 3 only includes a first automated image analysis display layout and a last automated image analysis display layout, one or more intermediate automated image analysis display layouts (not illustrated) may be generated.

A display layout that includes annotated imaging data may further include one or more images that are related to the image in which latent features were detected.

Latent features in the medical imaging data may be marked with indicia (e.g., tags, icons, arrows, pointers) to generate annotated imaging data. The annotated imaging data may include a study image that is annotated with indicia to generate an annotated image, an overlay including indicia that is displayed on top of a study image, and the like. In the display layout 340 and the display layout 350, respectively, a latent feature 364 and latent feature 368 are marked.

In the example of FIG. 3, the fourth display layout 340 includes an automated image analysis finding (e.g., first CAD RCC projection 342) along with images from first display layout 310 (e.g., LCC projection 314, RMLO projection 316, LMLO projection 318). In other aspects, one or more of the projections displayed with the CAD RCC projection 342 may be an image related to an image in which CAD detected a lesion (e.g., RCC projection 312). The fifth display layout 350 includes an automated image analysis finding (e.g., second CAD RCC projection 352) along with images from the second display layout 320 (e.g., LCC projection 324, RMLO projection 326, LMLO projection 328). In other aspects, one or more of the projections displayed with the CAD RCC projection 352 may be an image related to an image in which CAD detected a lesion (e.g., RCC projection 322).

The automated image analysis display layout(s) may include first image views (e.g., a first image series) of one or more of the medical images (e.g., mammography images) of the current healthcare study from the first image series. In implementations, the automated image analysis display layout(s) can be a repetition of the display layouts for displaying a sequence of first image views (e.g., a first image series) of one or more of the medical images of the current healthcare study from the first image series with an annotated imaging data overlay turned ON. In implementations, the last step of a second set of review steps can be a repetition of review steps of a first set of review steps with the annotated imaging data overlay turned ON.

The display manager (display manager 216) may change the GUI on the display device of the display component to display the automated image analysis display layouts (e.g., fourth display layout 340, fifth display layout 350) for the healthcare study, which includes images of the second image series, in the GUI on the display device after the displaying of the last display layout of the first image series of the healthcare study.

In an instance where the second data source does not include automated image analysis findings for the healthcare study, then the display manager (display manager 216) may change the GUI on the display device of the display component to display a notification to the user that indicates that there are no automated image analysis findings for the healthcare study. The display layouts may define a CAD-integrated reading protocol for the healthcare study and may include a number of review steps. The review steps may be associated with particular display layouts.

A display layout may include one or more indicators. For example, the third display layout 330 includes an indicator 360 that indicates that a next display layout (e.g., series, image) includes annotated imaging data, the fourth display layout 340 includes an indicator 362 that indicates that at least one of the images displayed includes annotated imaging data, and the fifth display layout 350 includes an indicator 366 that indicates that the current display layout is a last display layout of the CAD-integrated reading protocol.

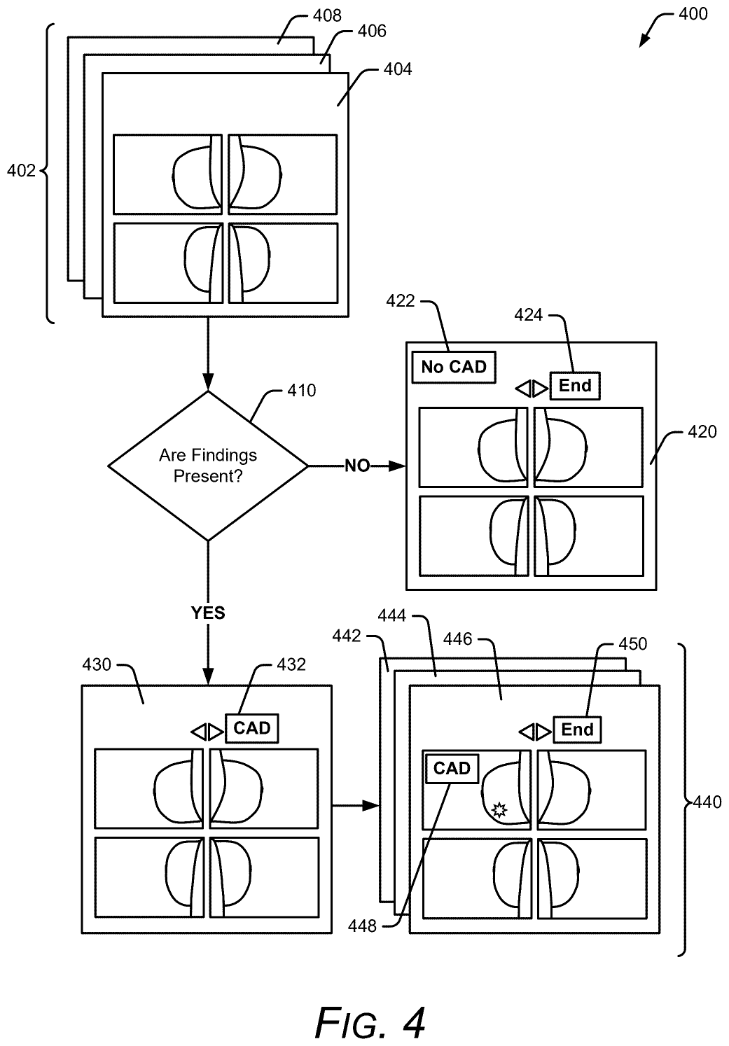

FIG. 4 illustrates an example process 400 for displaying healthcare study information, which may include medical imaging data from a current healthcare study and may also include one or more of legacy medical imaging data (e.g., study images from a previous healthcare study) or annotated imaging data. The process 400 is illustrated as a series of blocks that specify operations performed. The operations are not necessarily limited to the order or combinations shown for performing the operations by the respective blocks. Further, any of one or more of the operations can be repeated, combined, reorganized, or linked to provide a wide array of additional and/or alternate processes. In portions of the following discussion, reference can be made to the example system environment 100 of FIG. 1 and/or to entities or processes as detailed in FIGS. 1-3, reference to which is made for example only. The techniques are not limited to performance by one entity or multiple entities operating on one device. The operations illustrated and described herein are examples. The process 400 is similar to the process 300 illustrated in FIG. 3 and described above, except as detailed below.

As discussed with respect to the computing system 200 of FIG. 2, a study display module (e.g., study display module 202) receives selected healthcare study information for a current healthcare study from a data source (e.g., first database 230). The healthcare study information is selected by a selection component (e.g., selection component 212). Utilizing the selected healthcare study information, a layout generation component (e.g., layout generation component 214) generates study display layouts for displaying a sequence of first image views (e.g., a first image series) of one or more of medical images (e.g., mammography images) of the current healthcare study from the first image series. For example, the study display layouts may include a first display layout 404, a first intermediate display layout 406, a second intermediate display layout 408, and a last display layout (e.g., last display layout 420, last display layout 430). While FIG. 4 only includes two intermediate display layouts (e.g., first intermediate display layout 406, second intermediate display layout 408), in some implementations, additional intermediate display layouts may be presented. Further, an intermediate display layout may not be present. In FIG. 4, the first display layout 404 and the intermediate display layouts (e.g., first intermediate display layout 406, second intermediate display layout 408) are collectively represented as a display layout series 402.

The display layouts are for display in a GUI (e.g., GUI 250) of a display device of a display component (e.g., display component 218). The display layouts may include one or more current projections, legacy projections (e.g., legacy images from a previous healthcare study for a patient), or annotated projections including annotated imaging data. A display layout may include more than one type of projection to provide a side-by-side comparison of projections.

A display manager (e.g., display manager 216) receives the display layout(s) and generates a GUI on a display device of a display component (e.g., display component 218). In this way, the first display layout 404 is displayed to a clinician for review. After reviewing the contents of the first display layout 404, the clinician may provide (e.g., via input component 220) a user input to advance to a display of a next display layout. The display manager (e.g., display manager 216) then changes the GUI to display the next display layout of the healthcare study (e.g., the first intermediate display layout 406). After reviewing the contents of the first intermediate display layout 406, the clinician may provide a further user input to advance the display to a next display layout. The display manager (e.g., display manager 216) then changes the GUI to display the next display layout of the healthcare study (e.g., the second intermediate display layout 408). The process of displaying a display layout on the GUI and receiving user input from the clinician to advance the display to a next display layout may iteratively proceed until the clinician has viewed all display layouts of the display layout series 402.

After reviewing the contents of the display layout series 402, the clinician may provide (e.g., via input component 220) a further user input to advance the display to a next display layout. Upon receiving the user input to advance to a next display layout after the last display layout of the display layout series 402 has been displayed, the selection component (e.g., selection component 212) may determine (e.g., at operation 410) whether automated image analysis findings are present (e.g., stored in a second data source) for the healthcare study.

In an instance where automated image analysis findings are not present for the healthcare study (e.g., the second data source does not include automated image analysis findings for the healthcare study), then the display manager (e.g., display manager 216) may change the GUI on the display device of the display component to display a last display layout 420 from the first image series. The last display layout 420 may include an indicator 422 that indicates that there are no AI findings for the healthcare study and/or may include an indicator 424 that indicates that the current display layout is the last display layout of a CAD-integrated reading protocol. After advancing to the last display layout 420, the CAD-integrated reading protocol ends.

In an instance where automated image analysis findings are present for the healthcare study (e.g., the second data source includes automated image analysis findings for the healthcare study), then the display manager (e.g., display manager 216) may change the GUI on the display device of the display component to display a last display layout 430 from the first image series. The last display layout 430 may include an indicator 432 that indicates that automated image analysis findings (e.g., annotated imaging data) are present for the healthcare study.

Further, in an instance where automated image analysis findings for the healthcare study are present, the layout generation component (e.g., layout generation component 214) generates one or more automated image analysis display layouts for display in the GUI that includes automated image analysis findings (e.g., a sequence of second image views of images in the second image series). For example, in the process 400, the example sequence of display layouts further includes automated image analysis display layouts (e.g., first finding display layout 442, intermediate finding display layout 444, and last finding display layout 446) that include annotated imaging data. In FIG. 4, the finding display layouts are collectively represented as a finding display layout series 440.

The automated image analysis display layout may include annotated imaging data (e.g., medical imaging data in which findings (e.g., latent features) are marked). The annotated imaging data may include a study image that is annotated with indicia to generate an annotated image, an overlay including indicia that is displayed on top of a study image, and the like. For example, an automated image analysis display layout (e.g., finding display layout) may include projections from another display layout (e.g., projections from first display layout 404) overlaid with findings (e.g., annotated imaging data).

A display layout that includes annotated imaging data may further include one or more images that are related to an image in which latent features were detected. Latent features in the medical imaging data may be marked with indicia (e.g., tags, icons, arrows, pointers) to generate annotated imaging data. The annotated imaging data may include a study image that is annotated with indicia to generate an annotated image, an overlay including indicia that is displayed on top of a study image, and the like. The automated image analysis display layout(s) may include first image views (e.g., a first image series) of one or more of the medical images (e.g., mammography images) of the current healthcare study from the first image series. In implementations, the automated image analysis display layout(s) can be a repetition of the display layouts for displaying a sequence of first image views (e.g., a first image series) of one or more of the medical images of the current healthcare study from the first image series with an annotated imaging data overlay turned ON. In implementations, the last step of a second set of review steps can be a repetition of review steps of a first set of review steps with the annotated imaging data overlay turned ON.

After reviewing the contents of the display layout 430, the clinician may provide (e.g., via input component 220) further user input to advance the display to a next display layout. Upon receiving a user input to advance to a next display layout after the display layout 430 has been displayed, the display manager (display manager 216) may change the GUI on the display device of the display component to display an automated image analysis display layout of the finding display layout series 440 for the healthcare study, which includes images of the second image series, in the GUI on the display device. In this way, the automated image analysis display layout(s) are displayed after the display of the last display layout of the first image series of the healthcare study. For example, the clinician may provide a user input to advance from the display layout 430 to a next display layout and the display manager may change the GUI to display a first finding display layout 442. In this way, the first finding display layout 442 is displayed to the clinician for review. After reviewing the contents of the first finding display layout 442, the clinician provides (e.g., via input component 220) user input to advance the display to a next display layout (e.g., second finding display layout 444). The display manager (e.g., display manager 216) then changes the GUI to display the next display layout of the healthcare study. After reviewing the contents of the second finding display layout 444, the clinician provides (e.g., via input component 220) user input to advance the display to a next display layout (e.g., last finding display layout 446). The process of displaying a display layout on the GUI and receiving user input from the clinician to advance the display to a next display layout may iteratively proceed until the clinician has viewed all display layouts of the second image series of the healthcare study. After advancing to the last finding display layout 446, the CAD-integrated reading protocol ends.

An automated image analysis display layout (e.g., finding display layout) may include one or more indicators. The display layouts of the finding display layout series 440 may include an indicator 448 that indicates that one or more of the images displayed in the display layout include annotated imaging data (e.g., annotations). The layout for the last finding display layout 446 may include an indicator 450 that indicates that the current display layout is a last display layout of the CAD-integrated reading protocol.

### Example Processes

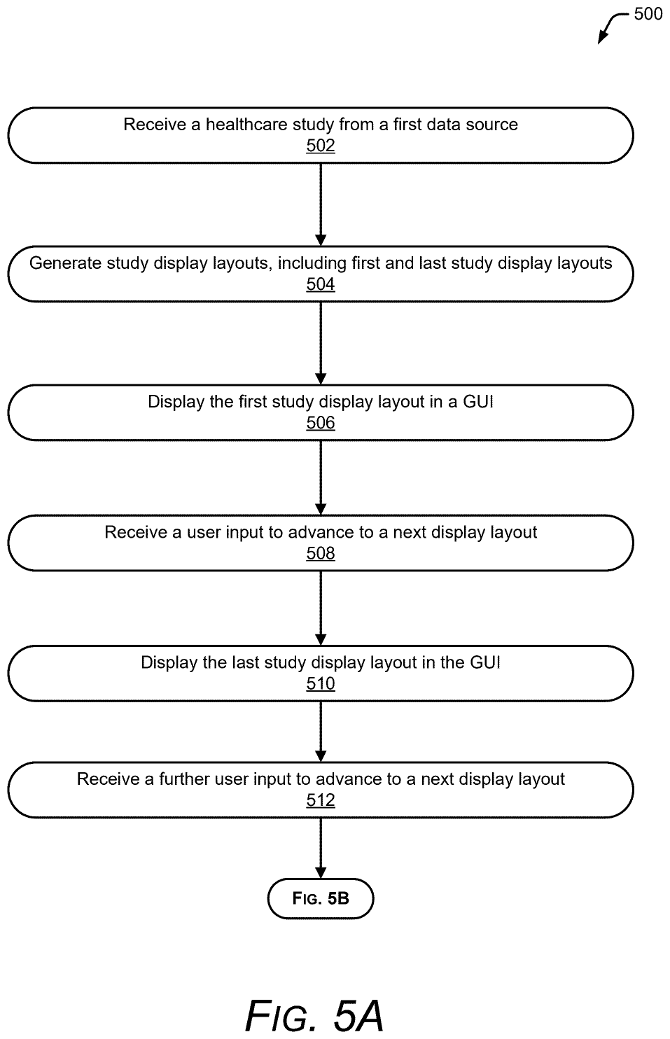

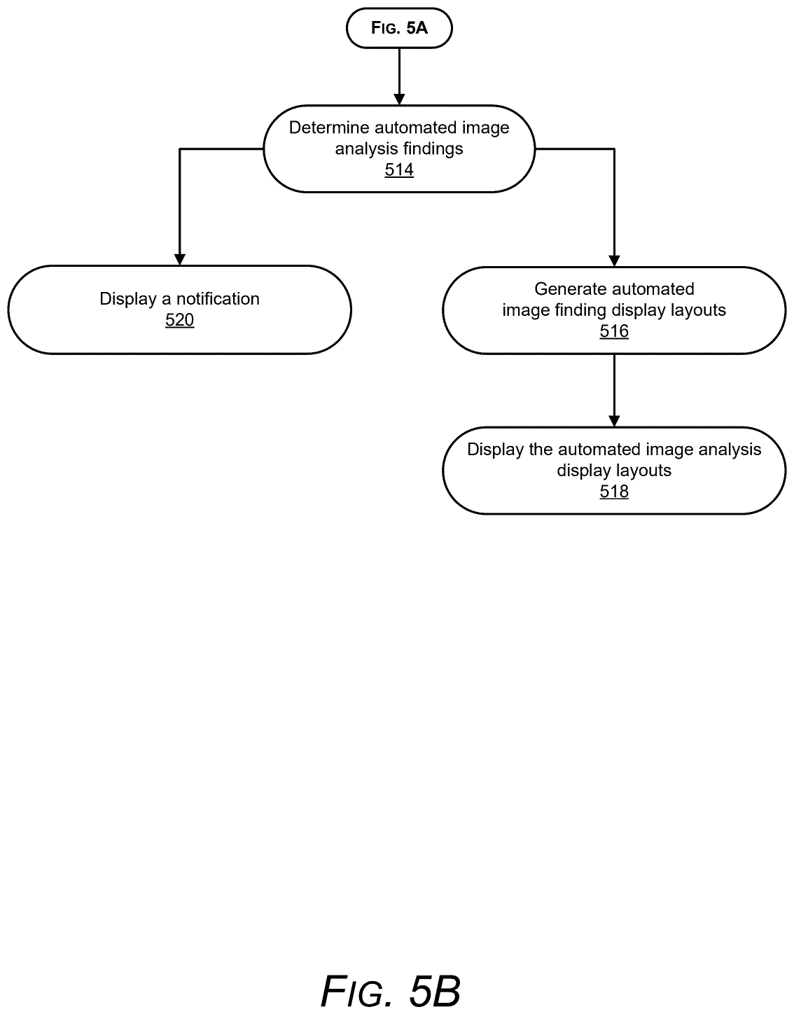

FIGS. 5A and 5B are a flow diagram of one implementation of a process 500 for displaying healthcare study information performed by a medical image management system (e.g., medical image management system environment 100 of FIG. 1, medical image management system 600). The process 500 is performed by processing logic that may include hardware (e.g., circuitry, dedicated logic), software (e.g., software run on a general-purpose computer system, software run on a dedicated machine), firmware, or a combination of two or more of these. The medical image management system may include a network communication interface that is configured to receive images of a healthcare study; an image cache memory to cache the images received; one or more processors coupled to the network connection interface and the memory and configured to display healthcare study information; and a display component (e.g., display screen) coupled to the one or more processors to display the images in a graphical use interface (GUI) of a display device.

The process 500 is shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example system environment 100 of FIG. 1 and/or to entities or processes as detailed in FIGS. 1-4, reference to which is made for example only. The techniques are not limited to performance by one entity or multiple entities operating on one device.

Referring to FIGS. 5A and 5B, the process 500 begins with the processing logic receiving a healthcare study (e.g., medical images) from a first data source (processing block 502). The healthcare study may include a first image series that includes more than one image. In aspects, the healthcare study is sent to the medical image management system by a modality that creates the study. In aspects, the healthcare study is sent from a medical image archive (e.g., a picture archiving and communication system (PACS) or other remotely located storage facility). In aspects, the healthcare study is received via a network interface and stored in a memory of the medical image management system.

The processing logic generates, from the first image series, study display layouts for display in a graphical user interface of a display device (processing block 504). The study display layouts include a first display layout and a last display layout. The processing logic displays the first display layout in the graphical user interface (processing block 506). The processing logic receives a user input to advance to the next display layout (processing block 508) and displays the last display layout in the graphical user interface (processing block 510). The processing logic receives a further user input to advance to the next display layout (processing block 512).

The processing logic determines whether a second data source includes automated image analysis findings for the healthcare study (processing block 514). In an instance where the second data source includes automated image analysis findings for the healthcare study, the processing logic generates an automated image analysis display layout for display in the graphical user interface from the automated image analysis findings and displays the automated image analysis display layout in the graphical user interface on the display device after the displaying of the last display layout of the first image series of the healthcare study (processing block 516). In an instance where the second data source does not include automated image analysis findings for the healthcare study, the processing logic displays a notification to the user in the graphical user interface that indicates that there are no automated image analysis findings for the healthcare study (processing block 518).

### Example Medical Image Management System

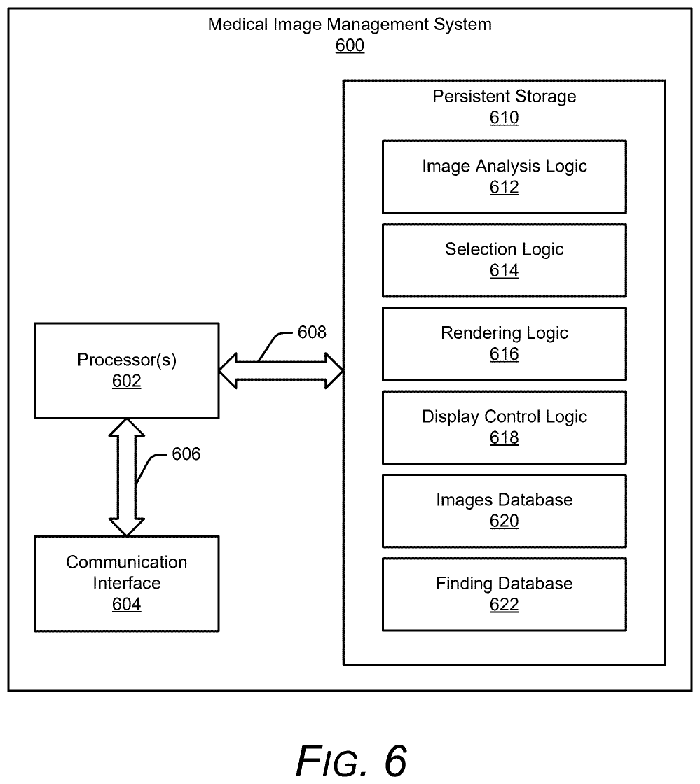

FIG. 6 illustrates an example aspect of a logical representation of a medical image management system 600 for displaying healthcare study information that is discussed above. The system 600 implements a CAD-integrated reading protocol to present healthcare study images and other relevant medical imaging data (e.g., annotated imaging data).

The medical image management system 600 may be implemented by one or more of the control server 110, a remote computer (e.g., remote computer 140), and/or the database cluster 120 of the medical image management system environment 100 discussed above with respect to FIG. 1. The medical image management system 600 includes one or more processors 602 that are coupled to communication interface logic 604 via a first transmission medium 606. The communication interface logic 604 enables communications with other electronic devices, specifically enabling communication with remote users such as doctors, nurses and/or medical technicians, remote databases (e.g., PACS) that store healthcare studies, and healthcare modalities that generate and send studies. According to one aspect of the disclosure, the communication interface logic 604 may be implemented as a physical interface including one or more ports for wired connectors. Additionally, or in the alternative, the communication interface logic 604 may be implemented with one or more radio units for supporting wireless communications with other electronic devices.

The medical image management system 600 performs a portion or all of the processing steps of systems and methods for displaying healthcare study information in response to processor(s) 602 executing one or more sequences of one or more instructions contained in a memory, such as persistent storage 610. Such instructions may be read into the persistent storage 610 from another computer-readable medium, such as a storage device. One or more processors in a multi-processing arrangement may also be employed to execute the sequences of instructions contained in the persistent storage 610. In aspects, hard-wired circuitry may be used in place of or in combination with software instructions. Thus, aspects are not limited to any specific combination of hardware circuitry and software.

As stated above, the medical image management system 600 includes at least one computer-readable medium or memory programmed according to the teachings of the disclosed systems and methods for displaying healthcare study information and for containing data structures, tables, records, or other data described herein. Examples of computer-readable media are compact discs, hard disks, floppy disks, tape, magneto-optical disks, programmable read-only memory (PROM) (e.g., erasable programmable read-only memory (EPROM), electrically erasable programmable read-only memory (EEPROM), Flash EPROM), dynamic random-access memory (DRAM), static random-access memory (SRAM), synchronous dynamic random-access memory (SDRAM), and the like. Stored on any one or on a combination of computer-readable media, the disclosed systems and methods for displaying healthcare study information include software for controlling the medical image management system 600, for driving a device or devices for implementing disclosed systems and methods, and for enabling the medical image management system 600 to interact with a human user. Such software may include, but is not limited to, device drivers, operating systems, development tools, and applications software. Such computer-readable media further include a computer program product of the disclosed systems and methods for performing all or a portion (if processing is distributed) of the processing performed in implementing the disclosed systems and methods for displaying healthcare study information. Computer code devices of the disclosed systems and methods for displaying healthcare study information may be any interpreted or executable code mechanism, including but not limited to scripts, interpreters, dynamic link libraries, Java classes, and complete executable programs. Moreover, parts of the processing of the disclosed systems and methods for displaying healthcare study information may be distributed for better performance, reliability, and/or cost. The term “computer-readable medium,” as used herein, refers to any medium that participates in providing instructions to processors 602 for execution. A computer-readable medium may take many forms, including but not limited to non-volatile media, volatile media, and transmission media.

The processor(s) 602 is further coupled to the persistent storage 610 via a transmission medium 608. The persistent storage 610 may store information and instructions to be executed by the processors 602. In addition, the persistent storage 610 may be used for storing temporary variables or other intermediate information during execution of instructions to be executed by processors 602. According to one aspect of the disclosure, the persistent storage 610 may include (a) image analysis logic 612, (b) selection logic 614, (c) rendering logic 616, (d) display control logic 618, (e) an images database 620, and (f) a findings database 622.

The image analysis logic 612 includes logic for performing analysis of images from healthcare studies (e.g., medical images). The image analysis may include activating one or more automated image analysis algorithms executing on one or more artificial intelligence (AI) engines and/or servers to produce findings (e.g., results, outputs, annotated imaging data) that are indicative of the results of the algorithm. The findings may include images, or portions thereof, from the analyzed healthcare study that are relevant to a diagnosis or condition of a patient. The image analysis logic 612 may perceive and mark latent features within medical imaging data (e.g., study images) to generate annotated imaging data. The image analysis logic 612 may utilize one or more indicia (e.g., tags, icons, arrows, pointers) to mark the latent features in the medical imaging data. The annotated imaging data may include a study image that is annotated with indicia to generate an annotated image, an overlay including indicia that is displayed on top of a study image, and the like. The annotated imaging data may be stored in a data source.

The selection logic 614 may include logic for selecting healthcare studies and accessing data sources (e.g., databases) to obtain the selected healthcare studies (e.g., retrieving one or more pieces of information from a storage device and importing each of the one or more pieces of information into a display area of a display layout). The pieces of information may include, but are not limited or restricted to, (i) medical images, including x-rays, mammograms, computerized tomography (CT) scans, magnetic resonance imaging (MRI), positron emission tomography (PET) scans, and/or ultrasound imaging, (ii) clinician notes regarding one or more of the medical images, and/or (iii) medical records corresponding to one or more of the subjects of the one or more medical images. The medical records may include other study data, for example medical parameter values (e.g., measurements, findings, impressions, patient demographics and history/risk factors) related to the healthcare study. The selection logic 614 may further determine whether a data source (e.g., database) includes automated image analysis findings (e.g., annotated imaging data) for a healthcare study and select, through access of the data source, the automated image analysis findings.

The rendering logic 616 includes logic for generating data for user interfaces, such as those, for example, described above. In one aspect, the rendering logic 616 performs one or more processing operations on data of healthcare studies to generate display data for displaying the content of the study, including any images and automated image analysis findings associated therewith. The rendering logic 616 may include logic for creating a display layout with one or more images from a series in a current study with images from series in one or more previously created studies.

The display control logic 618 receives the selected healthcare studies and automated image analysis findings (if present). The display control logic 618 renders the selected healthcare studies and automated image analysis findings in display layouts to implement the CAD-integrated reading protocol.

The images database 620 and the findings database 622 may include a single non-transitory computer-readable medium storage device or may each be a separate non-transitory computer-readable medium storage device. The images database 620 stores healthcare study information (e.g., medical images) for display in a display area of an image viewer or other GUI. The findings database 622 stores automated image analysis findings (e.g., images) for display in a display area of an image viewer or other GUI.

### Additional Examples

Some additional examples of systems and methods for displaying healthcare study information include the following Examples.
Example 1. A method comprising: receiving a healthcare study from a first data source, the healthcare study having a first image series that includes more than one image; generating, from the first image series, study display layouts for display in a graphical user interface of a display device, the study display layouts including a first display layout and a last display layout; displaying the first display layout in the graphical user interface; receiving a user input to advance to a next display layout; displaying the last display layout in the graphical user interface; receiving a further user input to advance to a next display layout; and determining whether a second data source includes automated image analysis findings for the healthcare study; in an instance where the second data source includes automated image analysis findings for the healthcare study, the method further comprising: generating, from the automated image analysis findings, an automated image analysis display layout for display in the graphical user interface; and displaying the automated image analysis display layout in the graphical user interface on the display device after the displaying of the last display layout of the first image series of the healthcare study; and in an instance where the second data source does not include automated image analysis findings for the healthcare study, the method further comprising: displaying a notification to a user in the graphical user interface that indicates that there are no automated image analysis findings for the healthcare study.
Example 2. The method of Example 1, further comprising: receiving the automated image analysis findings for the healthcare study from the second data source.
Example 3. The method of Example 2, further comprising: generating at least one automated image analysis finding indicator from the automated image analysis findings, wherein the automated image analysis display layout further comprises: at least one of the study display layouts overlaid with the at least one automated image analysis finding indicator.
Example 4. The method of Example 2, wherein generating the automated image analysis display layout for display in the graphical user interface from the automated image analysis findings further comprises: determining images of the first image series based on the automated image analysis findings; generating at least one automated image analysis finding indicator from the automated image analysis findings for each image of the first image series that has automated image analysis findings; and combining the generated automated image analysis finding indicators with the respective images of the first image series to generate images for the automated image analysis display layout.
Example 5. The method of Example 4, wherein generating the automated image analysis display layout for display in the graphical user interface from the automated image analysis findings further comprises: including copies of images of the first image series that do not have automated image analysis findings in the automated image analysis display layout.
Example 6. The method of Example 1, wherein the automated image analysis display layout includes at least one image of the first image series of the healthcare study.
Example 7. The method of Example 1, wherein the notification is displayed after the displaying of the last display layout.
Example 8. The method of Example 1, wherein the automated image analysis display layout comprises a plurality of automated image analysis display layouts.
Example 9. The method of Example 1, wherein the images of the first image series are mammography images.
Example 10. A medical image management system comprising: a network communication interface to receive healthcare studies; a memory coupled to the network communication interface to store received healthcare studies; a display device coupled to the memory to display the received healthcare studies; and one or more processors coupled to the network communication interface, the memory, and the display screen and configured to: receive a healthcare study from a first data source, the healthcare study having a first image series that includes more than one image; generate, from the first image series, study display layouts for display in a graphical user interface of the display device, the study display layouts including a first display layout and a last display layout; display the first display layout in the graphical user interface; receive a user input to advance to a next display layout; display the last display layout in the graphical user interface; receive a further user input to advance to a next display layout; determine whether a second data source includes automated image analysis findings for the healthcare study; in an instance where the second data source includes automated image analysis findings for the healthcare study: generate an automated image analysis display layout for display in the graphical user interface from the automated image analysis findings; and display the automated image analysis display layout in the graphical user interface on the display device after the displaying of the last display layout of the first image series of the healthcare study; and in an instance where the second data source does not include automated image analysis findings for the healthcare study: display a notification to a user in the graphical user interface that indicates that there are no automated image analysis findings for the healthcare study.
Example 11. The medical image management system of Example 10, wherein the one or more processors are further configured to: receive the automated image analysis findings for the healthcare study from the second data source.
Example 12. The medical image management system of Example 11, wherein the one or more processors are further configured to: generate at least one automated image analysis finding indicator from the automated image analysis findings, wherein the automated image analysis display layout further comprises: at least one of the study display layouts overlaid with the at least one automated image analysis finding indicator.
Example 13. The medical image management system of Example 10, wherein the one or more processors configured to generate the automated image analysis display layout for display in the graphical user interface from the automated image analysis findings are further configured to: determine images of the first image series with automated image analysis findings; generate at least one automated image analysis finding indicator from the automated image analysis findings for each image of the first image series that has automated image analysis findings; and combine the generated automated image analysis finding indicators with the respective images of the first image series to generate images for the automated image analysis display layout.
Example 14. The medical image management system of Example 13, wherein the one or more processors configured to generate the automated image analysis display layout for display in the graphical user interface from the automated image analysis findings are further configured to: include copies of images of the first image series that do not have automated image analysis findings in the automated image analysis display layout.
Example 15. Non-transitory computer-readable storage media having instructions stored thereupon, which, when executed by a system having at least one processor, a memory, and a display device therein, cause the system to perform a method comprising: receiving a healthcare study from a first data source, the healthcare study having a first image series that includes more than one image; generating, from the first image series, study display layouts for display in a graphical user interface of the display device, the study display layouts including a first display layout and a last display layout; displaying the first display layout in the graphical user interface; receiving a user input to advance to a next display layout; displaying the last display layout in the graphical user interface; receiving a further user input to advance to a next display layout; and determining whether a second data source includes automated image analysis findings for the healthcare study; in an instance where the second data source includes automated image analysis findings for the healthcare study, the method further comprising: generating, from the automated image analysis findings, an automated image analysis display layout for display in the graphical user interface; and displaying the automated image analysis display layout in the graphical user interface on the display device after the displaying of the last display layout of the first image series of the healthcare study; and in an instance where the second data source does not include automated image analysis findings for the healthcare study, the method further comprising: displaying a notification to a user in the graphical user interface that indicates that there are no automated image analysis findings for the healthcare study.
Example 16. The non-transitory computer-readable storage media of Example 15, further comprising receiving the automated image analysis findings for the healthcare study from the second data source.
Example 17. The non-transitory computer-readable storage media of Example 16, further comprising: generating at least one automated image analysis finding indicator from the automated image analysis findings, wherein the automated image analysis display layout further comprises: at least one of the study display layouts overlaid with the at least one automated image analysis finding indicator.
Example 18. The non-transitory computer-readable storage media of Example 15, wherein generating the automated image analysis display layout for display in the graphical user interface from the automated image analysis findings further comprises: determining images of the first image series with automated image analysis findings; generating at least one automated image analysis finding indicator from the automated image analysis findings for each image of the first image series that has automated image analysis findings; and combining the generated automated image analysis finding indicators with the respective images of the first image series to generate images for the automated image analysis display layout.
Example 19. The non-transitory computer-readable storage media of Example 18, wherein generating the automated image analysis display layout for display in the graphical user interface from the automated image analysis findings further comprises: including copies of images of the first image series that do not have automated image analysis findings in the automated image analysis display layout.
Example 20. The non-transitory computer-readable storage media of Example 15, wherein the automated image analysis display layout includes at least one image of the first image series of the healthcare study.

### CONCLUSION

In aspects, systems and methods for displaying healthcare study information may include one or more of the features of the systems and methods illustrated in the drawings and described above. Although implementations for systems and methods for displaying healthcare study information have been described in language specific to certain features and/or methods, the subject of the appended claims is not necessarily limited to the specific features or methods described. Rather, the specific features and methods are disclosed as example implementations of systems and methods for displaying healthcare study information.

What is claimed is:

1. A method comprising:
receiving a healthcare study from a first data source, the healthcare study having a first image series that includes more than one image;
generating, from the first image series, study display layouts for display in a graphical user interface of a display device, the study display layouts including a first display layout and a last display layout;
displaying the first display layout in the graphical user interface;
receiving a user input to advance to a next display layout;
displaying the last display layout in the graphical user interface;
receiving a further user input to advance to a next display layout; and
determining whether a second data source includes automated image analysis findings for the healthcare study;
in an instance where the second data source includes automated image analysis findings for the healthcare study, the method further comprising:
generating, from the automated image analysis findings, an automated image analysis display layout for display in the graphical user interface; and
displaying the automated image analysis display layout in the graphical user interface on the display device after the displaying of the last display layout of the first image series of the healthcare study; and
in an instance where the second data source does not include automated image analysis findings for the healthcare study, the method further comprising:
displaying a notification to a user in the graphical user interface that indicates that there are no automated image analysis findings for the healthcare study.

2. The method of claim 1, further comprising:
receiving the automated image analysis findings for the healthcare study from the second data source.

3. The method of claim 2, further comprising:
generating at least one automated image analysis finding indicator from the automated image analysis findings,
wherein the automated image analysis display layout further comprises:
at least one of the study display layouts overlaid with the at least one automated image analysis finding indicator.

4. The method of claim 2, wherein generating the automated image analysis display layout for display in the graphical user interface from the automated image analysis findings further comprises:
determining images of the first image series based on the automated image analysis findings;
generating at least one automated image analysis finding indicator from the automated image analysis findings for each image of the first image series that has automated image analysis findings; and
combining the generated automated image analysis finding indicators with the respective images of the first image series to generate images for the automated image analysis display layout.

5. The method of claim 4, wherein generating the automated image analysis display layout for display in the graphical user interface from the automated image analysis findings further comprises:
including copies of images of the first image series that do not have automated image analysis findings in the automated image analysis display layout.

6. The method of claim 1, wherein the automated image analysis display layout includes at least one image of the first image series of the healthcare study.

7. The method of claim 1, wherein the notification is displayed after the displaying of the last display layout.

8. The method of claim 1, wherein the automated image analysis display layout comprises a plurality of automated image analysis display layouts.

9. The method of claim 1, wherein the images of the first image series are mammography images.

10. A medical image management system comprising:
a network communication interface to receive healthcare studies;
a memory coupled to the network communication interface to store received healthcare studies;
a display device coupled to the memory to display the received healthcare studies; and
one or more processors coupled to the network communication interface, the memory, and the display screen and configured to:
receive a healthcare study from a first data source, the healthcare study having a first image series that includes more than one image;
generate, from the first image series, study display layouts for display in a graphical user interface of the display device, the study display layouts including a first display layout and a last display layout;
display the first display layout in the graphical user interface;
receive a user input to advance to a next display layout;
display the last display layout in the graphical user interface;
receive a further user input to advance to a next display layout;
determine whether a second data source includes automated image analysis findings for the healthcare study;
in an instance where the second data source includes automated image analysis findings for the healthcare study:
generate an automated image analysis display layout for display in the graphical user interface from the automated image analysis findings; and
display the automated image analysis display layout in the graphical user interface on the display device after the displaying of the last display layout of the first image series of the healthcare study; and
in an instance where the second data source does not include automated image analysis findings for the healthcare study:
display a notification to a user in the graphical user interface that indicates that there are no automated image analysis findings for the healthcare study.

11. The medical image management system of claim 10, wherein the one or more processors are further configured to:
receive the automated image analysis findings for the healthcare study from the second data source.

12. The medical image management system of claim 11, wherein the one or more processors are further configured to:
generate at least one automated image analysis finding indicator from the automated image analysis findings,
wherein the automated image analysis display layout further comprises:
at least one of the study display layouts overlaid with the at least one automated image analysis finding indicator.

13. The medical image management system of claim 10, wherein the one or more processors configured to generate the automated image analysis display layout for display in the graphical user interface from the automated image analysis findings are further configured to:
determine images of the first image series with automated image analysis findings;
generate at least one automated image analysis finding indicator from the automated image analysis findings for each image of the first image series that has automated image analysis findings; and
combine the generated automated image analysis finding indicators with the respective images of the first image series to generate images for the automated image analysis display layout.

14. The medical image management system of claim 13, wherein the one or more processors configured to generate the automated image analysis display layout for display in the graphical user interface from the automated image analysis findings are further configured to:
include copies of images of the first image series that do not have automated image analysis findings in the automated image analysis display layout.

15. Non-transitory computer-readable storage media having instructions stored thereupon, which, when executed by a system having at least one processor, a memory, and a display device therein, cause the system to perform a method comprising:
receiving a healthcare study from a first data source, the healthcare study having a first image series that includes more than one image;
generating, from the first image series, study display layouts for display in a graphical user interface of the display device, the study display layouts including a first display layout and a last display layout;
displaying the first display layout in the graphical user interface;
receiving a user input to advance to a next display layout;
displaying the last display layout in the graphical user interface;
receiving a further user input to advance to a next display layout; and
determining whether a second data source includes automated image analysis findings for the healthcare study;
in an instance where the second data source includes automated image analysis findings for the healthcare study, the method further comprising:
generating, from the automated image analysis findings, an automated image analysis display layout for display in the graphical user interface; and
displaying the automated image analysis display layout in the graphical user interface on the display device after the displaying of the last display layout of the first image series of the healthcare study; and
in an instance where the second data source does not include automated image analysis findings for the healthcare study, the method further comprising:
displaying a notification to a user in the graphical user interface that indicates that there are no automated image analysis findings for the healthcare study.

16. The non-transitory computer-readable storage media of claim 15, further comprising:
receiving the automated image analysis findings for the healthcare study from the second data source.

17. The non-transitory computer-readable storage media of claim 16, further comprising:
generating at least one automated image analysis finding indicator from the automated image analysis findings,
wherein the automated image analysis display layout further comprises:
at least one of the study display layouts overlaid with the at least one automated image analysis finding indicator.

18. The non-transitory computer-readable storage media of claim 15, wherein generating the automated image analysis display layout for display in the graphical user interface from the automated image analysis findings further comprises:
determining images of the first image series with automated image analysis findings;
generating at least one automated image analysis finding indicator from the automated image analysis findings for each image of the first image series that has automated image analysis findings; and
combining the generated automated image analysis finding indicators with the respective images of the first image series to generate images for the automated image analysis display layout.

19. The non-transitory computer-readable storage media of claim 18, wherein generating the automated image analysis display layout for display in the graphical user interface from the automated image analysis findings further comprises:
including copies of images of the first image series that do not have automated image analysis findings in the automated image analysis display layout.

20. The non-transitory computer-readable storage media of claim 15, wherein the automated image analysis display layout includes at least one image of the first image series of the healthcare study.
